(19) United States
(12) Patent Application Publication
(10) Pub. No.: US 2015/0340133 A1
(43) Pub. Date: Nov. 26, 2015

(54) HERMETIC FEEDTHROUGH, METHOD FOR PRODUCING A HERMETIC FEEDTHROUGH, PRINTED CIRCUIT BOARD AND SURGICAL INSTRUMENT

(71) Applicant: OLYMPUS WINTER & IBE GMBH, Hamburg (DE)

(72) Inventors: Sebastian JUNGBAUER, Hamburg (DE); Sebastian STUEHLE, Hamburg (DE); Martin WIETERS, Hamburg (DE); Alexander BRUSS, Hamburg (DE)

(73) Assignee: OLYMPUS WINTER & IBE GMBH, Hamburg (DE)

(21) Appl. No.: 14/819,896

(22) Filed: Aug. 6, 2015

Related U.S. Application Data
(63) Continuation of application No. PCT/EP2014/000269, filed on Feb. 3, 2014.

(30) Foreign Application Priority Data
Feb. 7, 2013 (DE) 10 2013 202 037.6

(51) Int. Cl. H01B 17/30; H05K 1/03; A61B 1/00; H05K 3/00; A61B 1/04; H02G 3/22; H05K 1/02

(52) U.S. Cl. CPC H01B 17/30; H02G 3/22; H05K 1/0346; H05K 1/0298; H05K 1/0245; H05K 1/028; H05K 3/0014; A61B 1/04; A61B 1/00124; H05K 2201/0154; H05K 2203/1327

(57) ABSTRACT

A hermetic feedthrough for a video endoscope for feeding electrical lines from a first partial space into a second partial space, the hermetic feedthrough including: a partition wall for hermetically sealing the two partial spaces, and a printed circuit board produced by a thin-film technique and in which the electrical lines are embedded in a plastic, is cast with a plastic compound in a mold and post-cured, wherein the plastic compound forms the partition wall.

### CROSS-REFERENCE TO RELATED APPLICATION

The present application is a continuation of PCT/EP2014/000269 filed on Feb. 3, 2014, which is based upon and claims the benefit to DE 10 2013 202 037.6 filed on Feb. 7, 2013, the entire contents of each of which are incorporated herein by reference.

BACKGROUND

1. Field

The present application relates to a hermetic feedthrough for a video endoscope for feeding electrical lines from a first partial space into a second partial space, in particular from a distally arranged and hermetically sealed housing into an endoscope shaft, comprising a partition wall for hermetically sealing the two partial spaces, a method for producing a hermetic feedthrough, a printed circuit board and a surgical instrument.

2. Prior Art

With video endoscopes, an optical system, for example an objective looking straight ahead or laterally, to which in many cases an image sensor or a pair of image sensors is connected, which convert the received light into electronic image information and forward it on proximally as electronic signals, is located on the distal tip of the endoscope shaft. Pairs of image sensors can be used for example for stereo video endoscopes for producing a spatial impression, for improving the color rendering, or for setting different sensitivities or for different analyses, for which different optical properties are needed.

The autoclavability of the endoscope is an important requirement. During autoclaving, the endoscope is treated with hot steam under high pressure. In the case of optical endoscopes and in particular video endoscopes, it is necessary to protect the optical components and the image sensor from steam which can otherwise condense on the lens system upon cooling and impair the optical quality of the system. Video endoscopes are therefore normally constructed in a hermetically sealed manner. The hermetic seal prevents steam from penetrating into the hermetically sealed region. With conventional video optical systems, this normally extends from the shaft tip into the handle.

In prior art video endoscopes, both the optical system and the image sensor are located in a hermetically sealed space. A hermetically sealed through-contact of these electrical and signal lines must thus be present. These electrical lines, with which among other things the electrical signals inside the endoscope shaft are transmitted, are normally cables with several shielded and/or unshielded stranded wires. With corresponding video endoscopes, the hermetically sealed through-contact takes place by means of metal pins or respectively metallic contact pins poured into glass. The electrical lines are soldered directly to the metal pins.

With optical systems having a sideways viewing direction, which can also be rotated about the longitudinal axis of the endoscope shaft, a rotation of the image sensor towards the sideways viewing optical system, for example a prism unit, and thus the jacket tube, is additionally necessary. The rotation of these two optical components against each other occurs in the hermetically sealed space. The image rotation is generated by the user in the handle of the optical system and must be transferred up to the tip. Thus, the seal must be guaranteed from the handle up to the tip of the video endoscope. In this case, the space in the jacket tube is thus limited and is utilized in order to implement a hermetically sealed unit, to transfer an image rotation, to transport light, and to guarantee a mechanically resilient design.

During the image rotation, the electrical lines are twisted between the image sensor or the image sensors and the hermetic feedthrough. The cable is thus soldered on the pins of the hermetic feedthrough such that its central axis is located on the rotational plane of the rotation. Since the cable is also frayed and the individual stranded wires are soldered on the hermetic connector, the cable can be twisted evenly with little force.

However, the soldering of the individual stranded wires on the individual contact pins is complex, error-prone, associated with high process risk and thus expensive to produce.

Because there are endoscopes with different lengths of the endoscope shafts and jacket tubes, a video-optical unit, i.e. the unit which comprises the optical system with object lens and the image sensor, must be produced for each endoscope. A modular design with different lengths, which could also be different nationally, is therefore expensive.

Besides the normally used plug-in connectors, which must be contacted on both sides in a manual soldering process with cables or printed circuit boards, and in which a hermetic sealing is then necessary through the soldering in of the plug housing, there are alternative known methods for feeding through signals with an at least sufficiently high impermeability, for example the gluing or respectively casting of cables or flexboard with adhesives. Experience has shown that these feedthroughs are usable in a restricted manner since moisture can get both between signal conductor and casting compound as well as between the casting compound and the hollow space to be sealed.

### SUMMARY

In contrast, an object is to provide a hermetic feedthrough, a method for its production and a surgical instrument, with which an improved sealing of hermetic housings and easy installability is given accompanied by good signal quality.

The object is solved through a hermetic feedthrough for a video endoscope for feeding electrical lines from a first partial space into a second partial space, in particular from a distally arranged and hermetically sealed housing into an endoscope shaft, comprising a partition wall for hermetically sealing the two partial spaces, which is further developed in that a printed circuit board, in particular a flexible printed circuit board, which is produced by a thin-film technique and in which the electrical lines are embedded in a plastic, is cast with a plastic compound in a mold and post-cured, wherein the plastic compound forms the partition wall.

In contrast to the previously used plug-in connectors, the printed circuit board produced by a thin-film technique is very simple and automatically contactable since its contact surfaces can be arranged in one plane. These thin-film printed circuit boards, which have a thickness of less than 100 μm, in most cases less than 50 μm, are highly flexible so that they are also twistable to a sufficient degree for rotation, for example for changing the viewing direction.

The corresponding selection of the plastics or of the plastic for the printed circuit board on one hand and the plastic compound for the partition wall on the other hand as well as the post-curing ensure a hermetic seal, since a completely surface-connected seal results. This results in a cost advantage through the forgoing of a separate hermetic connector, a simple adaptability to changed geometries and an increased electromagnetic compatibility through reduced electromagnetic radiation.

The plastic compound and the plastic of the printed circuit board can be made of the same curable material or different intercurable materials, in particular a polyimide. The plastic compound and/or the plastic of the printed circuit board can not yet be fully cured or hardened before the post-curing so that they can form an integral connection with each other as a result of the post-curing. Polyimide, but also other post-curable materials, offers or respectively offer material properties, which are suitable for proper use as a partition wall both in terms of stability and impermeability.

In a further embodiment, the printed circuit board and the plastic compound are casted into a ring, in particular a metal ring. The metal ring then serves to connect with the other parts of the surgical instrument for forming and for sealing the hermetically sealed housing. The housing can also be a separated part of the endoscope shaft itself. In this manner, the hermetic feedthrough can also be evenly dimensioned for hermetic housings with different lengths, i.e. for different endoscope types.

In order to prevent steam from getting through a gap between the partition wall and the ring, the ring can have an undercut, which is filled in by the plastic compound. The undercut, which is filled in by the plastic compound, thus forms a labyrinth seal, which effectively prevents steam from flowing through.

The object is also solved by a method for producing a hermetic feedthrough for a video endoscope, in which electrical lines are fed through a partition wall from a first partial space into a second partial space, which is further developed in that a printed circuit board, in particular a flexible printed circuit board, which is produced by a thin-film technique and in which the electrical lines are embedded in a non-conducting curable or cured or partially cured plastic, is cast with a non-conducting curable plastic compound in a mold and subsequently after treated for curing, in particular thermally. The type of the after treatment depends on the type of material selected.

This method is particularly suitable for producing a previously described hermetic feedthrough and leads to a hermetic feedthrough with integral seal between the printed circuit board and the partition wall, wherein the printed circuit board is particularly flexible and easily manageable.

The plastic compound and the plastic of the printed circuit board can be made of the same curable material or different intercurable materials, in particular of a polyimide.

The mold can be designed as a ring, in particular as a metal ring, wherein the hardened and/or cured plastic compound forms the partition wall in the ring, wherein in particular the ring has an undercut, which is filled in by the plastic compound during casting. These characteristics ensure that a hermetic seal is formed not only between the partition wall and the printed circuit board but also between the partition wall and the ring.

The object is further solved by a printed circuit board, in particular of a hermetic feedthrough for a surgical instrument, in particular a printed circuit board of a hermetic feedthrough previously described, produced by a thin-film technique with sequences of structured layers of metal and of a plastic, which is characterized in that the metal structures in cross-section through the printed circuit board produce a coaxial structure, wherein one or more, in particular one, two or four, conductors arranged in a center of the coaxial structure are surrounded around the entire perimeter by an, in particular one-piece or multi-piece, jacket conductor structure.

With the thin-film technique for producing the printed circuit board, complicated conductor structures can also be incorporated in layers into the surrounding plastic so that advantageous properties for signal transmission are realizable. The thin-film technique, can be, for example, a combination of the application of thin plastic layers and their processing, for example, through lithography and etching, wherein metal layers are applied, for example, through sputtering and subsequent photolithographical processing. The alternating application and processing of plastic and metal layers makes it possible to build up metal structures positioned in any manner, with up to 4 or 5 metal layers. Thus, for example, one, two, four or another number of printed circuit board tracks can be incorporated into a coaxial structure, in which the conductors arranged in the center of the structure are surrounded by a one-piece or multi-piece jacket conductor structure. The jacket conductor structure does not have to be circular, but can be rectangular or designed in another shape as long as it surrounds the inner-lying signal conductors. When the jacket structure is connected to ground, the inner-lying signal conductors are mainly protected from interference signals from outside.

It is provided in a further development that three or more layers of the printed circuit board are structured for forming a twisted conductor structure or a partially twisted conductor structure of two electrical conductors. A twisted conductor structure can be produced through corresponding masks in several planes, which generate conductor structures fitting on each other and continuing in several planes so that a type of “twisted pair” conductor is produced, which further reduces the impact of external interference signals on the signal quality. The signal conductors can also be passed around each other in another manner or engage in each other, e.g. in a “partial twisting,” without being completely twisted.

The object is also solved by a surgical instrument, in particular a video endoscope, with a hermetic feedthrough previously described. Such a surgical instrument can also have the printed circuit board previously described.

The advantages, characteristics and properties named for the individual subject matters, i.e. the hermetic feedthrough, the method, the printed circuit board (if it is part of the hermetic feedthrough), and the surgical instrument, also apply readily to the respective other subject matters.

Further characteristics will become apparent from the description of embodiments together with the claims and the included drawings. Embodiments can fulfill individual characteristics or a combination of several characteristics.

### BRIEF DESCRIPTION OF THE DRAWINGS

The embodiments are described below, without restricting the general idea of the invention, based on exemplary embodiments in reference to the drawings, whereby we expressly refer to the drawings with regard to the disclosure of all details according to the invention that are not explained in greater detail in the text. In the figures:

FIGS. 1a to c illustrate a schematized top view and two cross-sections through a hermetic feedthrough and printed circuit board,

FIG. 2 illustrates cross-sections and layer sequences for two printed circuit boards in a schematized representation,

FIG. 3 illustrates cross-sections, top views and layer sequences for a further printed circuit board in a schematized representation and

FIG. 4 illustrates cross-sections of a top view and layer sequences for a further printed circuit board in a schematized representation.

In the drawings, the same or similar types of elements and/or parts are provided with the same reference numbers so that a re-introduction is omitted.

### DETAILED DESCRIPTION

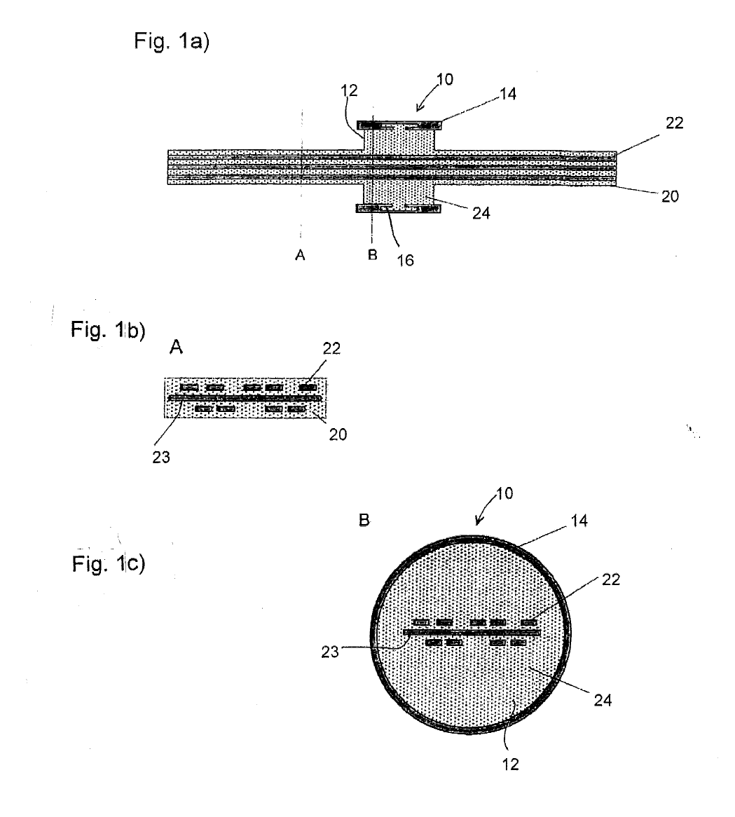

FIG. 1a) shows schematically a hermetic feedthrough 10 with a printed circuit board 20 in a cross-section. The elongated printed circuit board 20 has in the shown top view three printed circuit board tracks 22 surrounded by plastic, which end in open contact surfaces on both ends of the printed circuit board 20 (not shown). In the center, the printed circuit board 20 is surface-connected with a partition wall 12. For this, the separation layers between the printed circuit board tracks 22 and the partition wall 12 are made respectively of a plastic, for example a polyimide, which was post-cured after casting, for example through a thermal treatment.

The partition wall 12 is potted into a circumferential ring 14, for example a metal ring, which has an undercut 16 centrally and circumferentially on the inside, into which the plastic compound 24 of the partition wall 12 penetrates and which fills in this undercut 16. In the cross-section, the undercut 16 is T-shaped in the exemplary embodiment according to FIG. 1a).

The hermetic feedthrough 10 according to FIG. 1a) is easy to produce in that the prefabricated and at least already partially cured printed circuit board 20 is cast into the ring 14 together with the plastic compound 24 and is then post-cured. The plastic compound 24 and the plastic of the printed circuit board 20 are for example a polyimide, which is very suitable for this application.

It is ensured through the undercut 16 that a labyrinth seal results, which effectively prevents a diffusing through of steam if a gap should form between the plastic compound 24 of the partition wall 12 and the ring 14.

FIG. 1a) also shows two sectional planes A, B. The cut through the sectional plane A is shown in FIG. 1b), the cut through the sectional plane B in FIG. 1c).

FIG. 1b) shows a cut through the printed circuit board 20. It is shown that the printed circuit board 20 consists of a total of 7 layers, of which the first, third, fifth and seventh layer, counted from above, are made respectively entirely of plastic, for example polyimide, while the second, fourth and sixth layer are respectively printed circuit board tracks 22 or respectively a wide earth conductor 23. In this manner, the printed circuit board tracks 22 above and below the earth surface 23 are effectively decoupled from each other. All printed circuit board tracks 22 and the earth surface 23 are completely surrounded by the plastic in the shown sectional plane A.

In FIG. 1c), the cut is shown according to sectional plane B from FIG. 1a). The result of the method is that the printed circuit board 20 now blends in an integral manner, i.e. transition-less, into the partition wall 12 made of the plastic compound 24. This partition wall 12 reaches up to the circumferential ring 14. The undercut 16 is not shown in FIG. 1c).

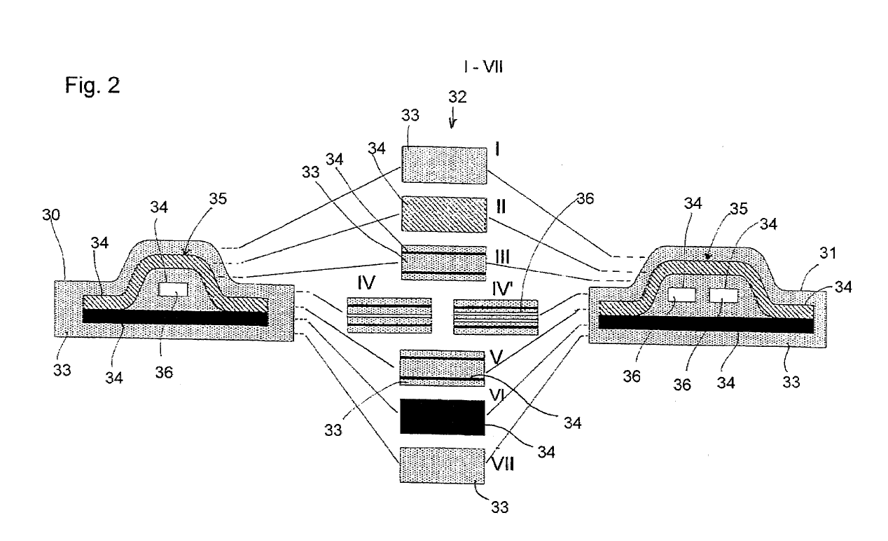

Different printed circuit board track structures for printed circuit boards produced by thin-film technology are then introduced in FIGS. 2 to 4.

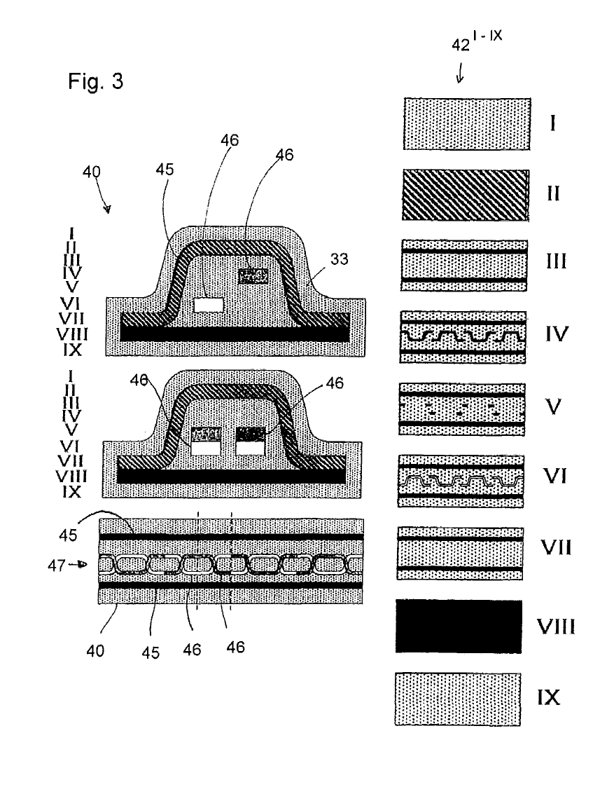

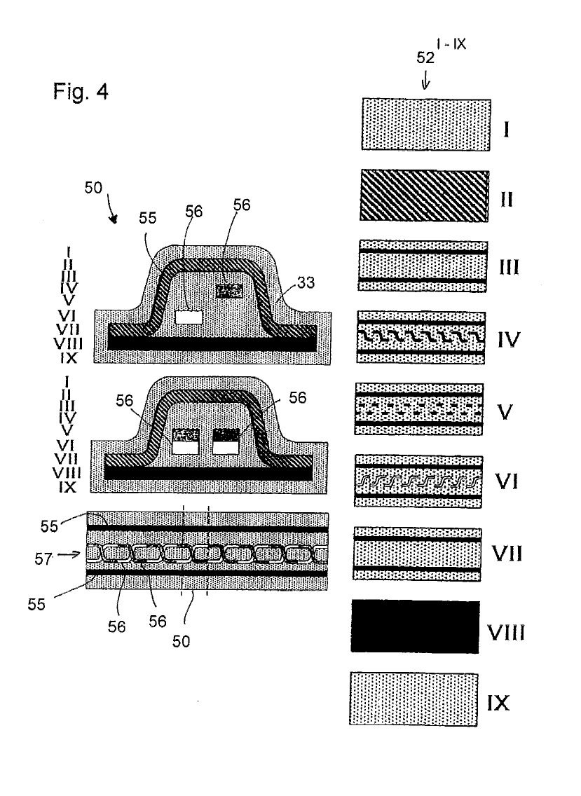

FIG. 2 shows two printed circuit boards 30, 31 in the left and right image parts, which differ in that the printed circuit board 30 has a signal conductor 36 and the printed circuit board 31 has two signal conductors 36. In both cases, the signal conductor or signal conductors 36 are surrounded by a jacket structure 35, which is also built in a layered manner. The entire thickness of these printed circuit boards 30, 31 as well as the printed circuit board 20 from FIG. 1 and the printed circuit boards shown in FIGS. 3 and 4 is less than 50 nm.

A sequence of structured layers 32^{I-VII} is shown in the center between the cross-sections through the printed circuit boards 30, 31. Each layer 32^{I-VII} relates to the corresponding layers in the cross-section of the printed circuit boards 30, 31 through corresponding lines, wherein the correspondence is greatly schematized since some layers are flat, while other layers can be contoured in height.

Since, in the production process, the structured layers 32^{I-VII} are built from bottom to top, they are shown below from bottom to top in relation to the printed circuit boards 30, 31.

The foundation is a flat layer 32^{VII}, which has the full width of the printed circuit boards 30, 31 and is made entirely of the used plastic material, for example polyimide. A structured layer 32^{VI}, which is made at least partially, centrally and completely of a metal, for example copper, nickel, gold or the like and has a good conductivity that is also flat is then applied to this. This layer 32^{VI} with the metal 34 forms the foundation or respectively a first part of a jacket structure 35 of a coaxial structure and is shown in a dark color. A one-piece structure of a jacket structure would make do without such a wide foundation layer and instead have a more circular or oval structure in cross-section.

The following flat structured layer 32^{V} is a plastic layer with two strip-like recesses, which are filled in after a few additional steps with a metal layer from the layer 32^{II}, which shows the upper, shaded part of the jacket structure.

In the following structured layer 32^{IV}, a metallic strip shown in a light color is embedded in the plastic as printed circuit board track 36, which is flanked by two recesses shown in a dark color, which also serve to form the upper part of the coaxial structure. Two signal conductors 36 of the printed circuit board 31 are present in the structured layer 32^{IV′}. This layer is replaced by the structured layer 32^{III}, which represents a repetition of the layer 32^{V}. All of these layers are mainly planar and have the same width.

The structured layer 32^{II} is a metallic layer, with which the jacket structure 35 is completed. This structured layer 32^{II} is not planar, but rather has a height distribution, since the metal, applied for example through sputtering, has penetrated into the recesses of the layers 32^{III-V}. The uppermost, covering, structured layer 32^{I}, which is a pure plastic layer, is also not planar. Thus, the entire metallic structure in the printed circuit boards 30, 31 is embedded in plastic on all sides.

The explanations about the structured layers according to FIG. 2 also mainly apply for the printed circuit boards according to FIGS. 3 and 4. However, since three-dimensional structures of the respective printed circuit board tracks are realized in these exemplary embodiments, the number of layers increases by two. The differences compared to FIG. 2 concern the three central structured layers, which contain the printed circuit board tracks. The corresponding reference numbers from FIG. 2 are increased by ten in FIG. 3, by 20 in FIG. 4.

FIG. 3 shows two cross-sections and a top view as well as a layer sequence of a printed circuit board 40. The Roman numbers Ito IX, which symbolize the different layers, are positioned to the side schematically in both cross-sections, in order to roughly illustrate their arrangement. It is a partially twisted structure, in which two signal conductors 46 are arranged partially, but not completely twisted. Moreover, the two signal conductors 46 are arranged in a jacket structure 45. The general structure of the layer structure and of the cross-section of the printed circuit board 40 approximately matches that in FIG. 2.

A difference results in the layers 42^{IV-VI}, in which the partially twisted structure is shown. In the uppermost layer, the dark parts of the signal conductors 46 are shown with black details, while, in the plane 42^{VI}, the lower plane with the lower parts of the signal conductors 46 is shown in white. A middle plane 42^{V} is inserted, which primarily stores plastic 33 at this position, which is electrically insulating, so that an electrical short circuit does not occur here. Only at the points where the signal conductors 46 are arranged laterally with respect to each other as seen in the longitudinal direction does the middle plane 42^{V} have through-contacts, with which the upper parts are connected with the lower parts of the signal conductors 46 so that a three-dimensional conductor structure results.

The two signal conductors 46 thereby respectively make a half rotation around each other and then reverse their direction so that a partially twisted conductor structure 47 is created. It has shielding properties similar to a fully twisted conductor structure.

Two sectional planes are shown in the top view, bottom left in FIG. 3. The cross-section on the top left in FIG. 3 thereby corresponds with the left cutting line and the middle cross-section on the left in FIG. 3 with the right cutting line.

FIG. 4 shows a printed circuit board 50 with reference numbers increased respectively by ten compared with FIG. 3, in which a fully twisted conductor structure 57 results. After each twist of a section of a signal conductor 56 from left to right, a through-contact is executed into the lower or respectively vice versa into the upper plane, i.e. from IV to VI and vice versa, which can also be seen in the tighter sequence of the through-contact points in the plane 52^{V}. A fully twisted conductor structure 57, which has the same shielding properties, in addition to the jacket structure 55, like a coaxially shielded “twisted pair” conductor, thus results. A very good signal quality is achieved in this manner.

It is further noted that the layers 42^{II, VIII}, 52^{II, VIII} in the FIGS. 3 and 4 as well as the layers 32^{II, VI} in FIG. 2 do not cross the complete width of the printed circuit boards 30, 31, 40, 50, but are rather also surrounded laterally by plastic 33 in the respective printed circuit board.

All named characteristics, including those taken from the drawings alone and also individual characteristics which are disclosed in combination with other characteristics are considered alone and in combination as essential for the invention. Embodiments can be realized by individual characteristics, or a combination of several characteristics.

### LIST OF REFERENCE NUMBERS

10 Hermetic feedthrough
12 Partition wall
14 Ring
16 Undercut
20 Printed circuit board
22 Printed circuit board track
23 Earth conductor
24 Plastic compound
30 Printed circuit board
31 Printed circuit board
32^{I-VII} Structured layers
33 Plastic
34 Metal
35 Jacket structure
36 Signal conductor
40 Printed circuit board
42^{I-IX} Structured layers
45 Jacket structure
46 Signal conductor
47 Partially twisted conductor structure
50 Printed circuit board
52^{I-IX} Structured layers
55 Jacket structure
56 Signal conductor
57 Fully twisted conductor structure

What is claimed is:

1. A hermetic feedthrough for a video endoscope for feeding electrical lines from a first partial space into a second partial space, the hermetic feedthrough comprising:
a partition wall for hermetically sealing the two partial spaces, and
a printed circuit board produced by a thin-film technique and in which the electrical lines are embedded in a plastic, is cast with a plastic compound in a mold and post-cured, wherein the plastic compound forms the partition wall.

2. The hermetic feedthrough according to claim 1, wherein the plastic compound and the plastic of the printed circuit board are made of the same curable material or different intercurable materials.

3. The hermetic feedthrough according to claim 1, wherein the printed circuit board and the plastic compound are cast into a ring.

4. The hermetic feedthrough according to claim 3, wherein the ring has an undercut, which is filled in by the plastic compound.

5. The hermetic feedthrough according to claim 1, wherein the printed circuit board is a flexible printed circuit board.

6. The hermetic feedthrough according to claim 2, wherein the plastic compound and the plastic of the printed circuit board is a polyimide.

7. The hermetic feedthrough according to claim 3, wherein the ring is a metal ring.

8. A method for producing a hermetic feedthrough for a video endoscope, in which electrical lines are fed through a partition wall from a first partial space into a second partial space, the method comprising:
producing a printed circuit board by a thin-film technique; the producing further comprising:
embedding the electrical lines in a non-conducting curable or cured plastic,
casting with a non-conducting curable plastic compound in a mold; and
subsequently aftertreating for curing.

9. The method according to claim 8, wherein the aftertreating is a thermal aftertreating.

10. The method according to claim 8, wherein the plastic compound and the plastic of the printed circuit board are made of the same curable material or different intercurable materials.

11. The method according to claim 10, wherein the plastic compound and the plastic of the printed circuit board is a polyimide.

12. The method according to claim 8, wherein the mold is designed as a ring, wherein the hardened and/or cured plastic compound in the ring forms the partition wall.

13. The method according to claim 12, wherein the ring is a metal ring.

14. The method according to claim 12, wherein the ring has an undercut, which is filled in by the plastic compound during casting.

15. A printed circuit board of a hermetic feedthrough for a surgical instrument, the printed circuit board comprising: sequences of structured layers made of metal structures and made of a plastic structures, wherein the metal structures in cross-section through the printed circuit board produce a coaxial structure, and one or more conductors are arranged in a center of the coaxial structure and are surrounded around an entire perimeter by a one-piece or multi-piece jacket conductor structure.

16. The printed circuit board according to claim 15, wherein the one or more conductors comprises two conductors.

17. The printed circuit board according to claim 15, wherein the one or more conductors comprises four conductors.

18. The printed circuit board according to claim 16, wherein three or more layers of the printed circuit board are structured for forming one of a twisted conductor structure or a partially twisted conductor structure of two electrical conductors.

19. A surgical instrument comprising a hermetic feedthrough according to claim 1.

20. The surgical instrument according to claim 19, wherein the surgical instrument is a video endoscope.
